(19) United States
(12) Patent Application Publication
(10) Pub. No.: US 2010/0256780 A1
(43) Pub. Date: Oct. 7, 2010

(54) Elastomeric stump sock for suspending by friction an artificial limb

(76) Inventor: Hansem So, Marion Station, MD (US)

Correspondence Address: Hansem So, 28461 St. Pauls Church Rd., Marion Station, MD, 21838, US

(21) Appl. No.: 12/384,319

(22) Filed: Apr. 2, 2009

(51) Int. Cl. A61F 2/80

(52) U.S. Cl. 623/36

(57) ABSTRACT

An elastomeric stump sock (10) with a high coefficient of friction that when donned grips the socket walls of an artificial limb. The only way to don and doff the artificial limb is by using a socket with a clamshell design. Using a donning sleeve (15), the stump is inserted into the socket (20A,20B) in the closed position. The socket (20A,20B) is opened for doffing.

### CROSS-REFERENCE TO RELATED APPLICATIONS

None.

### BACKGROUND

1. Field of Invention

This invention relates to stump socks, a.k.a., “liners,” specifically to such stump socks which are used to suspend an artificial limb from a stump of an amputated limb on a human being.

2. Description of Prior Art

In order to be useful, an artificial limb, a.k.a., prosthesis, must be attached to a human body at the site of an amputated limb, commonly called, “stump,” sometimes, “residuum.” Attaching the artificial limb to the stump was commonly referred to as, “suspension” or “prosthetic suspension.” Traditionally, the main means of achieving attachment was by suspending the artificial limb from the stump by belt, by vacuum, by suspension sleeve, or by, a most recent innovation, a pin-locking stump sock, commonly called, “pin-locking liner.”

All of these devices worked by making the socket part of the artificial limb hold on to the stump. The socket, a.k.a., “interface,” was basically a cup or a bucket attached to one end of the artificial limb. The socket was custom-shaped to match a shape of a stump of a particular person. The rest of the artificial limb, whether an arm or a leg, was attached to the distal or far end of the socket.

A fork strap knee-belt was a simple device, probably around since ancient times, attached to the top of the socket by rivets at one end and to the stump at the opposite end by a belt worn above the kneecap. There were variations involving a waistbelt, a metal hinge, elastic strap . . . and still other additional means of attachment. They all suffered from similar problems. The method was simple but crude, as the artificial limb was in essence tied or fastened to the stump with rope, strap, or hinge. The belt part of the devices had to be adjusted to the right length and even then suspension of the artificial limb from the stump was not even throughout the gait or walking cycle.

Vacuum suspension involved using a vacuum, or suction, socket. The advantage of the vacuum socket was that the socket was tightly stuck on the stump using suction. A long slippery sleeve, commonly called, “donning sleeve,” was donned over the stump and the stump was inserted into the socket. A slippery sleeve was necessary for insertion because the friction between the socket and skin would not allow insertion. The distal end of the sleeve, which often had a rope or strap attached to it, was inserted through a hole in a special vacuum valve at the bottom of the socket. The user pushed his or her covered stump down into the socket and at the same time yanked the donning sleeve out of the valve hole. When the stump was completely down in the socket, the user closed the valve hole by screwing in a threaded lid that blocked passage of air. The only way air could go into the socket once the valve was closed was through the top of the socket pass the stump.

In this manner, the socket was attached to the stump by vacuum. Of course, if the user made a sudden move or sat on a hard chair the vacuum often broke, air entered the socket through the top, and the artificial limb fell off. Furthermore, since the socket was held on to the stump by vacuum force, the skin was under constant suction. This contributed to edema; which was, to put it crudely, a really bad giant hickee. Edema was often followed by infection.

The suspension sleeve was basically a pliable hose made of neoprene or thermoplastic elastomer or other rubbery material. With the stump fully down in the socket, one end of the suspension sleeve encased and constricted the socket and the other end of the suspension sleeve encased and constricted the limb area above the stump. A person with a below-knee amputation (an amputation of the leg below the knee), for example, pulled the suspension sleeve over the knee and high up into the thigh area in order to achieve strong constriction over a large surface area for better suspension.

The user had to choose a suspension sleeve size small enough to constrict both the socket and the stump sufficiently enough to provide suspension. If the suspension-sleeve were too loose, either the socket, the stump, or both slipped out and the artificial limb fell off. The suspension sleeve was simple but it was hard to don, as the rubbery material had to be strong enough to provide the necessary level of constriction. A user with debilitated hand strength could not don and doff the suspension sleeve. Also, the necessarily strong constriction around the stump by the suspension sleeve was uncomfortable.

The above suspension methods have been around for decades and are still ubiquitous. Most recently, however, a pin-locking stump sock, commonly called, “pin-locking liner,” also became an ubiquitous means of suspending the artificial limb from the stump. This was a new innovation. The stump sock had been around for decades, if not ages, but had never been used for suspension of an artificial limb from the stump of an amputated limb.

Traditionally, the stump sock was donned over the stump before insertion of the stump into the socket. As with socks for our feet, the stump sock provided cushion and absorbed sweat. The stump sock also was used to control volume fluctuation of the stump. The amputated stump, especially a newly amputated one, routinely underwent volume change. Usually, the stump was bigger and swollen in the morning and as the day progressed got smaller as circulation removed excess bodily fluid from the stump. Addition or subtraction of thickness or number of layers of stump socks was used to control volume change so that the socket would fit tightly all day long. If the socket were loose, the artificial limb attached to it also was loose on the stump, and, like an untied shoe, was hard to use.

The pin-locking stump sock was made of two basic parts: the pin and the stump sock. A third part, a concave metal disk, was used to mount the pin on the closed end of the stump sock. The stump sock of the pin-locking liner, unlike the traditional stump sock, which was made of wool or fabric, was made of an elastomeric material, such as thermoplastic elastomer, silicone, or urethane rubber. U.S. Pat. No. 5,258,037 to Carl A. Caspers (1993) and U.S. Pat. No. 6,964,688 to Bruce G. Kania (2005) were but two variations of elastomeric stump socks. Sometimes, the elastomeric stump sock was also made to be used just as a sock, without the pin.

Unlike the wool or fabric stump sock, the sock part of the pin-locking stump sock, being composed of an elastomeric material, naturally possessed a high co-efficient of friction. Since elastomeric material are stretchy and rubbery by nature as well, the stump sock of the pin-locking stump sock, once donned, gripped the stump and was immovable. The traditional wool or fabric stump sock, if tugged on, would slide off the stump; just as a sock for our feet would slide off if tugged on. The friction between the stump and the elastomeric stump sock was so great, however, the pin-locking stump sock could not be removed by tugging on it. It had to be rolled off by curling the top edge down and inverting the stump sock. Likewise, in order to don it the user had to invert it and roll it onto the stump.

The pin was a piece of metal shaped like an arrow or rod. The pin was attached to the stump sock so that the pin pointed straight away from the closed end of the stump sock. After donning the pin-locking stump sock, the user aimed the pin towards a hole in a shuttle lock at the bottom of the socket. Put there by a technician. The user then inserted the pin into the shuttle lock by pushing down with his or her stump. The pin had serrations or teeth on it which enabled the pin to be locked into the internal mechanism of the shuttle lock.

The shuttle lock was designed so that the pin, once inserted into it, could not come back out unless the user pressed a release button. In this manner, the artificial limb was suspended from the stump via the socket, the shuttle lock, the pin, the stump sock, and, of course, the stump. This new method of suspension is ubiquitous today.

This new method of suspending or interfacing an artificial limb with the human body, however, brought with it new problems for artificial limb users. Since the entire weight of an artificial limb was suspended or dangled from a single metal pin, the stretching of the stump sock on the skin inevitably caused pain. This pain was especially pronounced for many above-knee amputees (people with a limb amputated above the knee) because of the sensitivity of the thigh area.

Another problem with the pin-locking stump sock was that the elastomeric stump sock part of it was made with a slippery outer surface. Slipperiness was usually achieved by encasing the elastomeric stump sock in a slippery fabric, such as polyester, nylon, or lycra. Some pin-locking stump socks had no fabric cover but the outer surface was specially altered at the factory to be slippery. Without slipperiness a pin-locking stump sock could not be used because the user could never get his or her stump into the socket. The friction between the elastomer and the socket would have blocked insertion.

Unfortunately, making the outer surface of the elastomeric stump sock slippery brought with it problems. The stump pistoned up-and-down and rotated inside the socket during walking. The artificial limb became unpredictable. The artificial foot or knee was prone to suddenly turning when the user stepped on an uneven surface. Pistoning made the user less able to judge the level of the ground surface. Pistoning also meant the weight of the artificial limb was dangling harder off the pin and causing more pain in the manner mentioned.

To remedy this problem, manufacturers often added felt to the outer surface of the stump sock to increase friction. In other words, first they made it slippery, then they tried to reduce the slipperiness to prevent pistoning and rotation. Of course, however, they could not increase the friction too much because then the user could not insert the stump into the socket. Thus the pin-locking stump sock remained slippery.

### SUMMARY

In accordance with the present invention a stump sock for enclosing an amputated limb made of an elastomeric material with surface coefficient of friction high enough on its outer surface facing an artificial limb socket wall to suspend an artificial limb.

### Objects and Advantages

Accordingly, the aim of the current invention is
(a) to provide a stump sock that suspends an artificial limb from a stump without pistoning or rotation during walking;
(b) to provide a stump sock that provides suspension without stretching on the skin;
(c) to provide a stump sock that provides suspension by friction between the socket;
(d) to provide a stump sock that protects the stump from direct suction force.

### DRAWING FIGURES

FIG. 1 shows a cross-sectional frontal view of the elastomeric stump sock. This stump sock has only one layer.

FIG. 2 shows the stump with the elastomeric stump sock donned over it. The donning sleeve and bivalve socket are shown.

FIG. 3 shows cross-sectional frontal view of the other preferred embodiment of the elastomeric stump sock. This stump sock has two layers.

FIG. 4 shows an isometric view of the elastomeric stump sock. This stump sock has one layer.

### REFERENCE NUMERALS IN DRAWINGS

10 elastomeric stump sock

15 donning sleeve

20A main part of bivalve socket

20B door part of bivalve socket

40 vacuum valve

50 socket lock

60 inner layer

65 outer layer

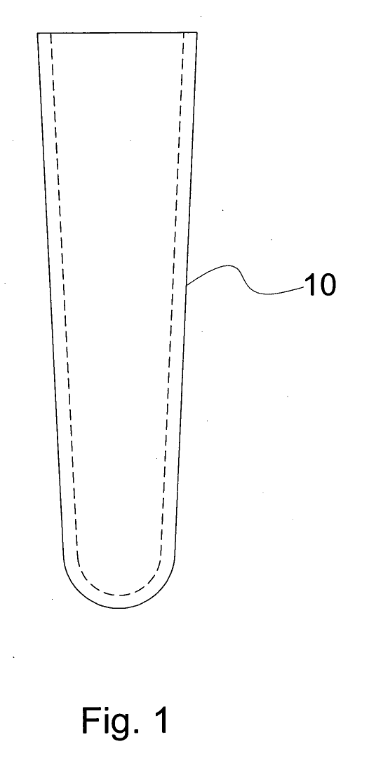

### DESCRIPTION—FIGS. 1,2,4—MOST PREFERRED EMBODIMENT

The present invention composition comprises any elastomer or synthetic rubber including but not exclusive to thermoplastic elastomer, urethane rubber, silicone-based elastomer, polymeric gel . . . or any other form of elastomeric material or synthetic rubber appropriate for use in the medical industry.

The present invention composition comprises any elastomeric material but does not provide any fabric or chemical covering or surface treatment to the most outer surface of the stump sock (10), on the side of the stump sock (10) opposite from the stump when donned, so as to cause a decrease in the coefficient of friction.

However, a presently most preferred embodiment of the present invention comprises an elastomer composed of thermoplastic elastomer. Kraton G1654 was used but any appropriate thermoplastic elastomer would have sufficed.

The G1654 was blended with mineral oil to produce a gel that was in the durometer (Shore A) of 1-20 range. That range is comfortable for the skin. A mold was prepared and the stump sock was injection molded.

In another most preferred embodiment, silicone resin was poured into a negative mold. The positive mold was then inserted into the negative mold and fixated in a position so that the gap between the positive core mold and the negative mold was the desired thickness and contour. The mold was then placed in a vacuum chamber to prevent air bubbles from forming in the stump sock. When the resin had set, the outer negative mold was opened and the positive mold was pulled out.

### Operation of the Most Preferred Embodiment—FIGS. 1, 2, 4

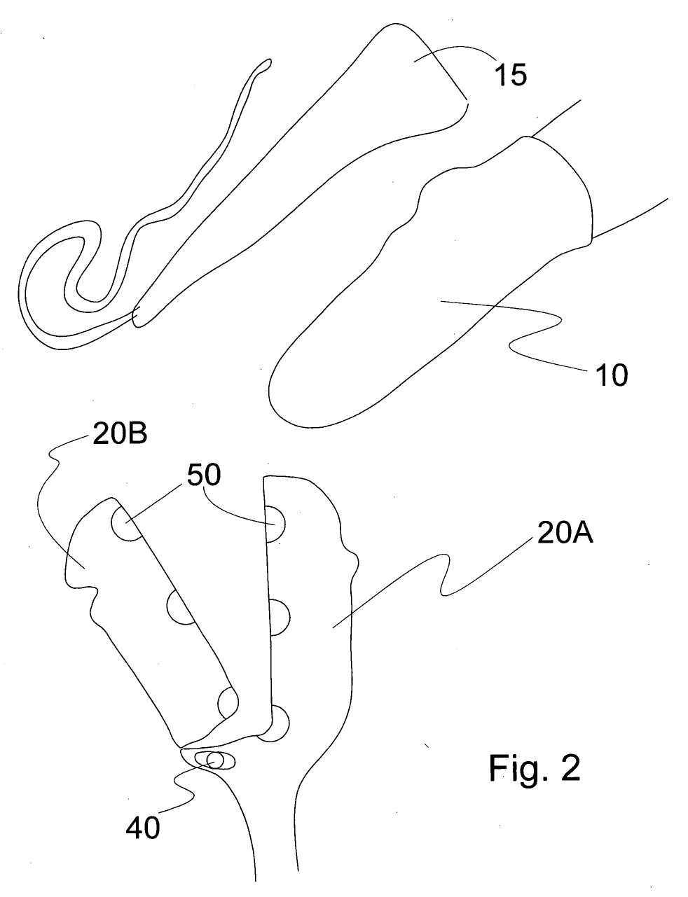

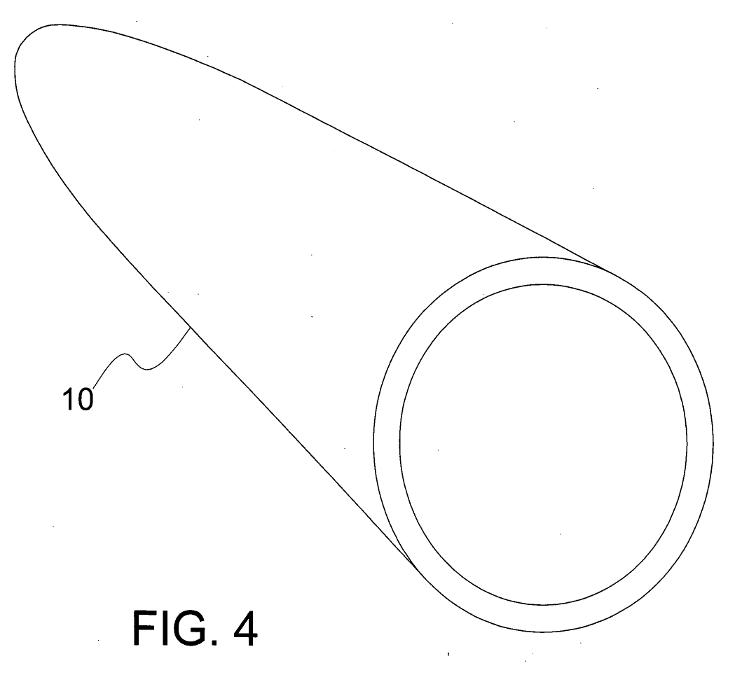

FIG. 1 shows a frontal cross-section illustration of the presently most preferred embodiment of the stump sock (10) of this invention. FIG. 4 shows an isometric view of the stump sock (10) with the open end in the foreground and the closed end in the background. The artificial limb socket must be of a bivalve, or clamshell, design, as illustrated in FIG. 2, or some other design that allows the socket to open and close. FIG. 2 shows the bivalve socket (20A, 20B) with the door (20B) partially open. The bivalve artificial limb socket was invented for the present invention but no patent is sought. The bivalve design is ubiquitous in various types of orthopedic appliances.

The user dons the stump sock (10) in the usual way by rolling it onto his or her stump. First, the bivalve socket door (20B) is closed and locked shut using locks (50). Then, the user inserts the stump with the elastomeric stump sock (10) donned on it into the socket using the conventional vacuum valve (40) and donning sleeve (15) method that has been around for decades.

In this method, the elastomeric stump sock (10) is rolled onto the stump. Then, the donning sleeve (15), a.k.a., “donning sock,” is donned over the elastomeric stump sock (10). The distal end of the sleeve (15), which usually ends in a rope or strap, as shown in FIG. 2, is inserted through a hole in the vacuum valve (40) at the bottom. The user pushes the stump down into the socket (20A,20B) and at the same time yanks the sleeve (15) out of the hole (40). The user continues to milk and squeeze the stump down into the socket (20A,20B), as is customarily done, while pulling out the donning sleeve (15). When the stump is completely down in the socket (20A,20B), the user closes the hole in the valve (40), usually with a threaded lid, so that no air could go through. The only way air could go into the socket (20A,20B) once the valve (40) is closed is through the top of the socket (20A,20B) pass the stump. In this manner, the socket (20A,20B) is attached to the stump by natural vacuum.

Once the elastomeric stump sock (10) has been inserted in this manner into a socket (20A,20B) the naturally high friction of the elastomer against the inner surface of the socket (20A,20B) prevents the stump from exiting the socket (20A,20B). The elastomer also clings to the skin of the stump and compresses the stump in general because of its elasticity. Also, since the stump was squeezed and milked down into the socket (20A,20B) with force, as normally done to make the socket fit tight and to pump excess bodily fluid out of the stump, the stump is pressed against the wall of the socket (20A,20B). In this manner, the artificial limb is securely suspended or attached to the stump during walking and, in the case of upper-extremity (arm) artificial limbs, other activities requiring limb movement.

Amputated limbs, or stumps, normally undergo volume change throughout the day. Usually, the stump is bigger in the morning and as the day progresses it gets smaller as fluid exits the stump. Traditionally, the user added wool or fabric stump socks to the stump to take up the slack. With the elastomeric stump sock (10) of the present invention, in order to accommodate volume change in the stump, the user adds or subtracts extra layers or plys of elastomeric stump sock (10). The user simply rolls on another stump sock (10) on top of the one s/he already has on.

### EXAMPLE

Mr. Jones is an upper-extremity amputee. He lost his right arm a couple of inches above the elbow in an accident. Every morning, Mr. Jones gets up and dons his artificial limb. He rolls on his elastomeric stump sock (10). Then, he closes the door (20B) on the socket (20A,20B) and locks the locks (50). The door (20B) may or may not be held on to the socket (20A) with a hinge or strap.

After closing and locking shut his socket (20A,20B), Mr. Jones opens the vacuum valve (40) at the bottom of his socket (20A,20B). He dons a donning sleeve (15), such as the commercially available E-Z Don, over his stump sock (10) and inserts the distal or far end on his donning sleeve (15), usually comprising a rope or strap, into the socket (20A,20B) and out of the valve (40). He then pulls on the rope with his hands from outside of his socket (20A,20B). At the same time, he pushes his stump down into the socket (20A,20B). As he does so, he gradually yanks out the donning sleeve (15) until the stump is completely down in the socket (20A,20B) and the donning sleeve (15) has completely exited the socket (20A,20B) through the valve (40). Assured that the distal end of his stump has reached the bottom of the socket (20A,20B), he closes the vacuum valve (40) and puts his donning sleeve (15) in his golf bag. He makes sure he has extra stump socks (10) in his golf bag so that later in the afternoon when his stump shrinks he can add more stump socks (10).

Mr. Jones goes to the golf driving range for practice. Now, when he swings his golf club the friction, in addition to the vacuum, between the socket (20A, 20B) and the elastomeric stump sock (10) and also between his elastomeric stump sock (10) and his stump keep his stump from rotating or pistoning in the socket (20A,20B). Mr. Jones plays golf with more confidence.

Mr. Jones comes home after a great day of golf. Now he must doff his artificial limb so he can shower. He unlocks the locks (50) and fully opens the socket door (20B) and easily pulls his stump out of the socket (20A, 20B). He rolls off, that is, doffs, all layers of the elastomeric stump sock (10), cleans them and puts them away.

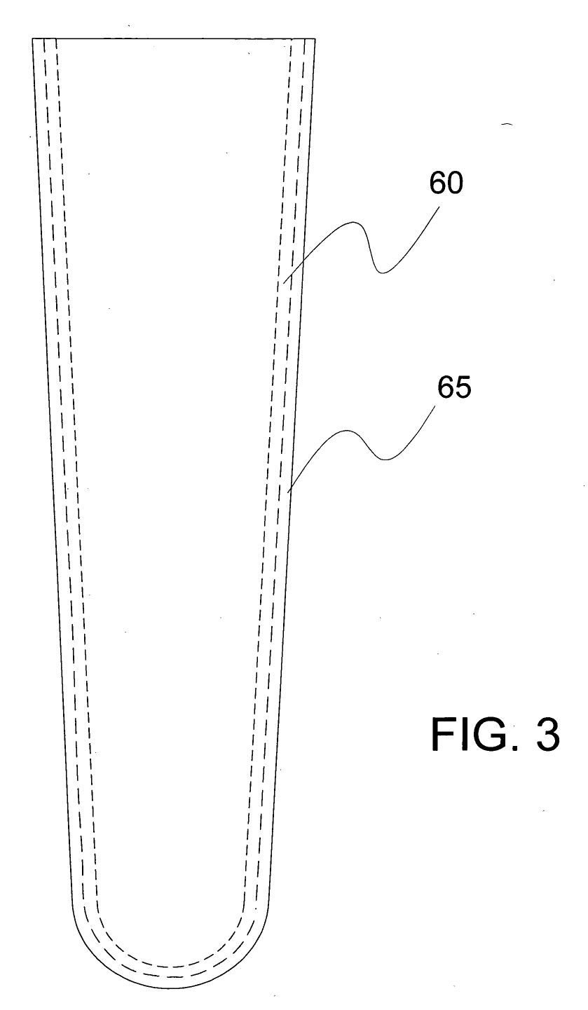

### Other Preferred Embodiments—FIG. 3

Furthermore, FIG. 3 shows a longitudinal cross-section of another preferred embodiment and variation of the friction stump sock (10) of this invention. An outer layer (65) composed of a higher durometer, preferrably Shore A 10-30, and an inner layer (60) composed of a softer durometer, preferrably Shore A 1-20.

A mixture of melted Kraton G1654 and Duoprime 70 mineral oil was prepared. A mold was dipped into the molten blend and removed. An elastomeric inner layer (60) was the result and was ready for step two.

The inner layer (60) was dipped again into another batch of molten blend of a higher durometer the same way. The result was an elastomeric stump sock (10) but with two layers (60,65) of thermoplastic elastomer blend instead of one. The outer layer (65) had a higher durometer for more durability and the inner layer (60) which goes against the skin had a lower durometer for comfort.

In yet another preferred embodiment, a layer of stretchable spandex fabric sewn into a matching stump sock shape was inserted between the two layers (60,65). The result was a stump sock (60,65) with stretchable spandex fabric sandwiched between two layers of elastomer blend.

In still yet another preferred embodiment, the inner layer (60) and the outer layer (65) was made separately so that the two layers may be separable and modular.

### Operation of Other Preferred Embodiments—FIG. 3

The user dons and uses the multi-layered stump sock (60,65) in the same way as the single-layered stump sock (10) in the most preferred embodiment. If the inner layer (60) and the outer layer (65) are modular and separable, the user simply dons the inner layer (60) and then dons the outer layer (65) over the inner layer (60). Doffing is simply the reverse of donning.

### Conclusion, Ramifications, and Scope

Accordingly, the reader will see that the elastomeric stump sock of this invention can be used to attach an artificial limb to the stump of an amputated limb without pistoning or artificial foot or knee rotation and without harmful direct suction on the skin of the stump.

Accordingly, the scope of the invention should be determined not by the embodiment(s) illustrated, but by the appended claims and their legal equivalents.

What is claimed as new and desired to be secured by Letters Patent of the United States is:

1. A stump sock comprising an elastomer with a surface coefficient of friction sufficiently high enough to make infeasible to use as a daily routine the donning and doffing of a hard, rigid, non-partitioned, non-clamshell, traditional resin laminate suction socket of an artificial limb without any use of a donning sleeve or any other assistive means whatsoever.

2. The claim of 1 wherein said elastomer comprises chemically or physically altered composition with further increased coefficient of friction.

3. The claim of 1 wherein said elastomer comprises chemically or physically treated outer surface with further increased coefficient of friction.

4. The claim of 1 wherein said elastomer comprises fabric surface or cover with a coefficient of friction comparable to said elastomer.

5. The claim of 1 wherein said elastomer comprises silicone.

6. The claim of 1 wherein said elastomer comprises urethane rubber.

7. The claim of 1 wherein said elastomer comprises thermoplastic elastomer.

8. The claim of 1 wherein said elastomer comprises thermoset elastomer.

9. The claim of 1 wherein said elastomer comprises mineral oil.

10. The claim of 1 wherein said elastomer comprises one or more layers of elastic fabric material sandwiched between two or more layers of elastomer.

11. The claim of 1 wherein said elastomer comprises two or more layers of elastomer of varying durometer or hardness.

12. The claim of 1 wherein said elastomer comprises two or more layers of elastomer of varying thickness.

13. The claim of 11 wherein said layers are separable and modular from each other.

14. The claim of 1 wherein said elastomer comprises localized sections of elastomer of varying durometer or hardness.

15. The claim of 1 wherein said elastomer comprises localized areas of elastomer of varying thickness.

16. The claim of 1 wherein said elastomer comprises a foamed or non-foamed gel composition comprising a block co-polymer.

17. The claim of 1 wherein said elastomer comprises skin-enhancing additives.

18. The claim of 1 wherein said stump sock comprises a tube-shaped covering for enclosing a stump of an amputated limb, said covering comprising an open end for introduction of said stump and a closed end opposite said open end.

19. The claim of 1 wherein said stump sock comprises accommodative contouring for an amputated limb.

20. A method for suspending an artificial limb from a stump of an amputated limb comprising the steps of,
providing a stump sock, said stump sock providing an elastomeric material providing a coefficient of friction too high to sustain as a daily activity the routine donning and doffing of a rigid, hard, singularly unified, non-bivalve, traditional resin laminate vacuum socket on an artificial limb without the use of a donning sock or any other assistive means of any form, method, or substance.
